(19) United States
(12) Patent Application Publication
(10) Pub. No.: US 2005/0032760 A1
(43) Pub. Date: Feb. 10, 2005

(54) Continuous process for the production of r-rofleponide

(76) Inventor: John Gregg Van Alsten, Portage, MI (US)

Correspondence Address: PHARMACIA & UPJOHN, 301 HENRIETTA ST, 0228-32-LAW, KALAMAZOO, MI, 49007, US

(21) Appl. No.: 10/495,692

(22) PCT Filed: Feb. 9, 2004
(86) PCT No.: PCT/IB04/00407

(30) Foreign Application Priority Data
Feb. 12, 2003 (US) 60449254

(51) Int. Cl. A61K 31/56; C07J 5/00

(52) U.S. Cl. 514/177; 552/582

(57) ABSTRACT

A continuous process for the preparation of 16α,17α[(R)butylidenedioxy]-6α,9α-difluoro-11β,21-dihydroxypregn-4-ene-3,20-dione (rofleponide) is disclosed.

### CROSS REFERENCE TO RELATED APPLICATIONS

This application is the national phase of international application PCT/IB04/000407, which claims the benefit of U.S. provisional application Ser. No. 60,449,254, filed 21 Feb. 2003, under 35 USC 119(e)(i).

### BACKGROUND OF THE INVENTION

R-Rofleponide is a steroid that is useful for treating irritable bowel syndrome. R-Rofleponide has high anti-inflammatory, immunosuppressive and anti-anaphylactic activity. Thalen, et al., Steroids, 63, 37-43 (1998), report a synthesis of both R and S-Rofleponide and a chromatographic separation. EP 0 262 108 discloses a method of controlling the epimeric distribution in the preparation of Rofleponide in which the product is produced by transacetylation in a hydrocarbon solvent or a halogenated hydrocarbon solvent together with a hydrohalogen acid or an organic sulphonic acid as a catalyst and in the presence of small grains of an inert material reaction medium. A preferred acid for use in this process is perchloric acid. When the reaction is conducted in a halogenated hydrocarbon solvent, the 22R/22S epimeric distribution can be varied within the range of 40:60-60:40.

U.S. Pat. No. 5,939,409 discloses a transacetalization process for the production of rofleponide.

U.S. Pat. No. 4,404,200 discloses the production of R and S epimers of rofleponide that are separated by chromatography.

U.S. patent application Ser. No. 10/247,246 discloses a process for the preparation of rofleponide in which the starting acetonide, 6α, 9α-difluoro-11β,21-dihydroxy-16α, 17α-(isopropylidenedioxy)pregn-4-ene-3-20 dione is dissolved in a solvent such as methylene chloride. Preferably about three moles of butyraldehyde are added, followed by perchloric acid. Several moles of perchloric are used for each mole of the acetonide. After approximately 15 minutes a precipitate forms.

One disadvantages of all batch processes involving perchloric acid catalysis is that the initial precipitate is a rofleponide perchloric acid complex that includes both the desired steroid and several moles of perchloric acid per molecule of steroid. Tests have shown that this material is capable of explosion and upon explosion would release a thermal energy of approximately 3.4 kilojoules per gram, or about 75 percent of the energy released upon the explosion of trinitrotoluene. Batch processes for the production of rofleponide that involve the use of perchloric acid to enhance the R/S ratio produce substantial quantities of a potentially explosive substance. Accordingly, it would be desirable to develop a process that produces reasonable quantities of rofleponide, while maintaining the amount of the explosive complex that is present at any one time at a low level.

### SUMMARY OF THE INVENTION

Surprisingly, we have found that rofleponide, that is, 16α,17α[(R)butylidenedioxy]-6α,9α-difluoro-11β,21-dihydroxypregn-4-ene-3,20-dione (Formula II) may be produced by reacting dihydroflucinolone acetonide (DFCA), that is, 6α,9α-difluoro- 11β,21-dihydroxy-16α,17α-(isopropylidenedioxy)pregn-4-ene-3,20 dione (Formula I) with butyraldehyde and perchloric acid in a continuous process. A small percentage of the S isomer (FORMULA III) is produced as a side product of this reaction. In this process DFCA is dissolved in a suitable solvent and mixed with butyraldehyde. This mixture is pumped to a reactor tube where it is mixed with perchloric acid. The mixture proceeds through a reaction tube to the crystallizer where there is an approximately 10 minutes residence time before the material in the crystallizer is pumped to a quenching bath.

### DETAILED DESCRIPTION

Surprisingly, we have found that rofleponide, that is, 16α,17α[(R)butylidenedioxy]-6α,9α-difluoro-11β,21-dihydroxypregn-4-ene-3,20-dione (Formula II) may be produced by reacting dihydroflucinolone acetonide (DFCA), that is, 6α,9α-difluoro-11β,21-dihydroxy-16α,17α-(isopropylidenedioxy)pregn-4-ene-3,20 dione (Formula I) with butyraldehyde and perchloric acid in a continuous process. A small percentage of the S isomer (FORMULA III) is produced as a side product of this reaction.

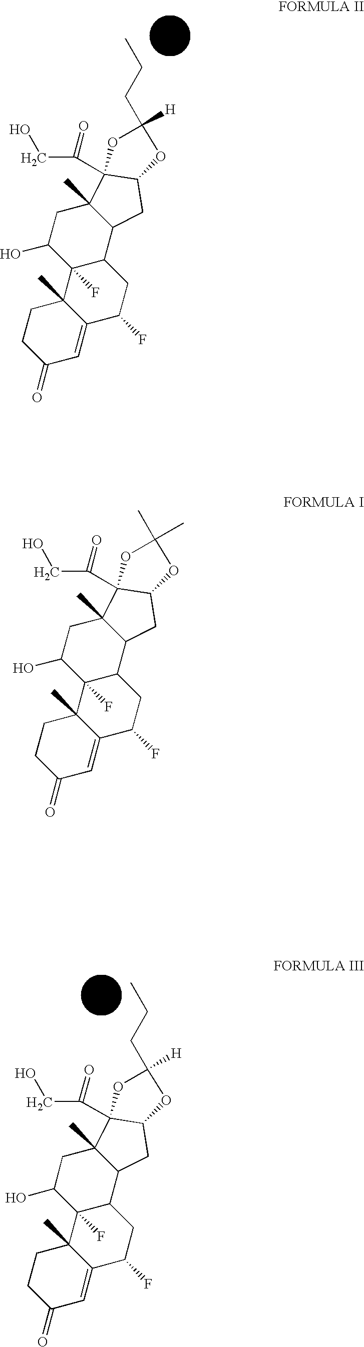

In the continuous process, the dihydroflucinolone acetonide is dissolved or suspended in a suitable solvent. The solvent can be any suitable liquid, preferably a liquid saturated alkane or a halogenated alkane, such as methylene chloride. An excess of butyraldehyde is added to dihydroflucinolone acetonide solution. These can range from 1.1 to 5 moles of butyraldehyde per mole of dihydroflucinolone acetonide. A preferred range is 2 to 4 moles of butyraldehyde per mole of dihydroflucinolone acetonide. A ratio of 3 moles of butyraldehyde per mole of dihydroflucinolone acetonide provides good results.

The perchloric acid used in the process is in the form of an aqueous solution with a concentration ranging from 60 to 71 weight percent. The perchloric acid is pumped to the reactor at a rate such that the ratio of the molar amount of perchloric acid to the molar amount of dihydroflucinolone acetonide is in the range of 2 to 6. A preferred range is 3 to 6 moles of perchloric acid per mole of dihydroflucinolone acetonide. A range of 5 to 6 moles of perchloric acid per mole of dihydroflucinolone acetonide gives good results.

It is important to assure that the stream of liquid containing the dihydroflucinolone acetonide achieves intimate mixing with the perchloric acid reactant. This mixing may be accomplished by mechanical agitation, static mixers or the use of turbulent flow within the reactor to achieve mixing. In one sense, the selection of the reactor is not critical. The reaction can take place in a vessel of almost any size and shape in which the appropriate mixing can be accomplished. The man skilled in the art could readily select an appropriate reaction vessel. However, to best achieve the purpose of the present invention, that is to prepare rofleponide while having a minimum amount of the explosive rofleponide perchloric acid complex present at any one time, it is preferred to conduct the reaction in a small diameter tube or pipe. The size of the pipe depends upon the amount of rofleponide desired. It is desirable to have a pipe such that at the flow rates being used, there will be turbulent flow within the tube or pipe. This allows turbulent mixing within the pipe without requiring the use of additional mixing equipment. The use of turbulent mixing offers several advantages. First, if the reactor is a small diameter tube, turbulent flow can be achieved with relatively small flow rates. The low flow rates allow the selection of a crystallizing vessel of small volume, and thereby assure that the volume of explosive intermediate that is present at any one time is relatively small. Typically, a reactor pipe could range from 1/16 to 1 inch in inside diameter. The length of the pipe or tube should be such that there is between 1 and 20 minutes residence time for the mixture in the reactor before it gets to the crystallizer.

Selection of pumps for the pumping of the dihydroflucinolone acetonide solution and the perchloric acid are rather important. If small diameter tubing and turbulent is used in order to have turbulent mixing, the liquids will suffer a rather large pressure drop while traveling through the reactor tube and, accordingly, pumps will be needed that are capable of low flow rates at high pressure. Diaphragm pumps may be used for this purpose, although the pulsating nature of these pumps makes them less than ideal. It has been observed that the pulsation of such pumps leads to poor mixing in the reactor. Gear pumps provide smoother flow and perform better in this process. Other pumps such as progressive cavity pumps, positive displacement pumps, peristaltic pumps, or centrifugal pumps are suitable for use in the present invention. It is also important to assure that the pumps maintain a reasonably constant output. The mole ratio of butyraldehyde to dihydoflucinolone is determined when the two are combined in a single solution. However, the mole ratio of perchloric acid to dihydoflucinolone is determined by the output volume of the respective pumps.

The crystallizer is a vessel large enough to contain slightly more than the volume of material that will flow through the system in approximately 10 minutes. The exact size of the crystallizer is not critical since the adjustment of the draw off level controls the amount of the rofleponide perchloric acid complex that is contained in the reactor. The draw off level is set at such a point that the residence time of the rationed product in the crystallizer is approximately 10 minutes. While the setting of 10 minutes as a reference time is convenient, the person skilled in the art will appreciate that crystallization is fast enough so that a short residence time is all that is required. During the residence time of the reaction product in the crystallizer, the most important thing that happens is that the R-rofleponide perchloric complex crystallizes. The crystallizer may be made or lined with a material that is inert to perchloric acid. Glass and stainless steel are examples of material that are suitable for the inner surface of the crystallizer. The rofleponide perchloric acid complex is sticky and may be difficult to remove from the surface of the crystallizer if the surface is not smooth enough. This problem may be solved with glass surfaces by treating the glass with alkyltrichlorosilanes wherein the alkyl group contains 1 to 16 carbon atoms. It is preferred that the alkyl groups have 8-16 carbon atoms. However, after such treatment, a glass crystallizer may be so smooth that the rofleponide perchloric acid complex may not nucleate on the walls of the reactor. In this case it may be necessary to add an externally produced seed crystal to the crystallizer. Once the crystallizer has been properly nucleated, it will function in a continuous process.

The quench bath is designed to break up the rofleponide perchloric acid complex. The quench bath contains either water or a two-phase mixture of water and suitable solvent such as methylene chloride or a C₅ to C₁₀ hydrocarbon solvent, e.g. isooctane. The rofleponide may be removed from the quench bath in a convenient manner and treated with aqueous base to remove the last traces of perchloric acid. The rofleponide may then be recrystallized from suitable solvents such as acetone/heptane or methylene chloride/branched octanes. The reaction is illustrated in the following SCHEME I: 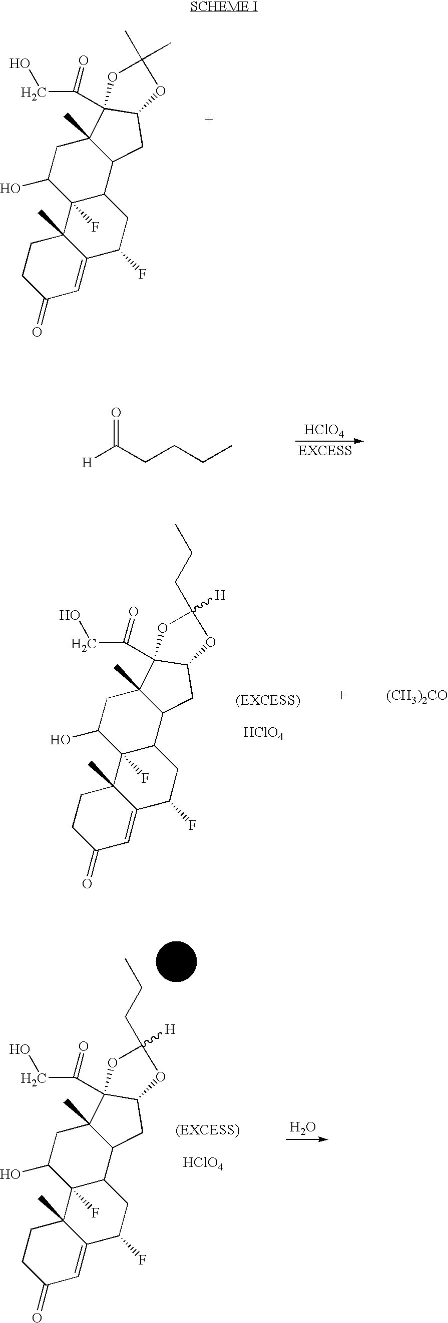 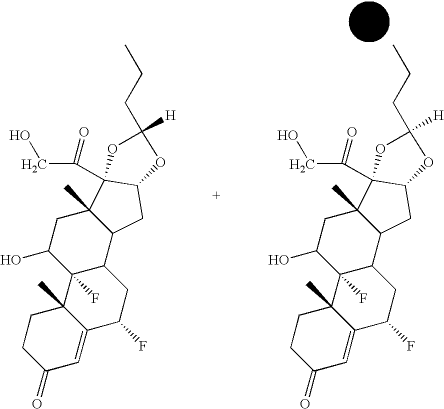
An example of a practical reactor to run the reaction is illustrated in SCHEME II. In this scheme methylene chloride has been selected as the solvent, and the term “XTALIZR” refers to the chamber in which crystallization occurs.

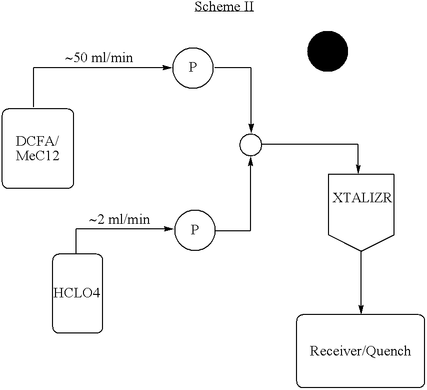

### EXAMPLE 1

The crystallizer was prepared by beginning a small-scale batch reaction within it. 14.1 gms (0.031 moles) of DFCA and 6.75 gms of butyraldehyde (0.093 moles), and 250 mls of methylene chloride were charged to the 2-liter glass vessel and stirred to dissolve at 23 C. 12.5 gms (0.087 moles) of 70% perchloric acid in water was added. Within 10 minutes solids were observed within the crystallizer.

56.5 gms (0.124 moles) of DFCA and 27.0 gms (0.375 moles) of butyraldehyde were dissolved together in 750 mls of methylene chloride. This solution was fed to pump A (“Gamma5”, diaphragm type, from Prominent, Inc.). 70% perchloric acid in water was fed to pump B (“GammaL”, diaphragm type, from Prominent, Inc.). Pump A was operated at a rate of approximately 55 mls/min (0.0081 moles DFCA/min), and pump B at ca. 2.2 mls/min (0.026 moles/min). The two streams were combined in a tee junction, then the two-liquid phase mixture traversed through a reaction element consisting of ca. 115 m of 1/16″ ID tubing. The outlet of the reaction element was fed to the crystallizer. The crystallizer was equipped with a drawoff at the 500 ml mark, which sent the intermediate slurry into a quench tank consisting of a mixture of methylene chloride and water. The slurry dissolved on mixing with the contents of the quench tank. The product-containing methylene chloride phase was analyzed by HPLC (Luna C18(2) column, 150×4.6 mm, using a mobile phase of 90/10 ethanol/water at 0.9 ml/min). The product mixture contained 5.0% of the DFCA starting material, 90.0% Rofleponide-“R”, and 4.0% Rofleponide-“S”.

### EXAMPLE 2

70.6 gms (0.155 moles) of DFCA and 33.75 gms (0.468 moles) of butyraldehyde were dissolved together in 1 liter of methylene chloride at 23 C. 250 mls of this solution was transferred to the crystallizer, and 20.7 gms (0.14 moles) of 70% perchloric acid added. Solids were observed to form within 10 minutes of the perchloric acid charge.

The balance of the steroid solution was fed by pump A as in Example 1, but at a rate of ca. 43 mls/min (0.0060 moles DFCA/min). 70% perchloric acid was fed by pump B, but at a rate of ca. 3 mls/min (0.034 moles/min). The two streams were combined, reacted, crystallized, and quenched as in Example 1. HPLC analysis showed that the product mixture was 0.3% DFCA, 98.2% Rofleponide-“R”, and 1.5% Rofleponide-“S”.

Branched octanes (250 mls) were then added to the quenced mixture, and the organic phase was then separated and washed with aqueous sodium bicarbonate to pH 9. The organic was separated from the bicarbonate solution and the solvent removed by vacuum distillation. The resulting solids were recrystallized from methylene chloride/branched octanes to yield Rofleponide in 93% yield, with 0.31% DFCA, 98.25% Rofleponide-“R”, and 1.45% Rofelponide-“S”.

### EXAMPLE 3

14 gms of DFCA (0.031 mole) and 66 gms of butyraldheyde (0.91 moles) were charged to the crystallizer and dissolved in 230 mls of methylene chloride at 23 C. 24 gms (0.17 moles) of 70% perchloric acid was charged to this mixture. A two phase mixture of oil in clear liquid was formed. After two hours in this state the crystallizer was seeded with a small amount of the solid Rofleponide intermediate. This resulted in the rapid conversion of the oil into solid.

700 gms of DFCA (1.54 moles) and 340 gms of butyraldehyde (4.72 moles) were dissolved in 11.5 liters of methylene chloride at 23 C. This solution was fed by pump A1 (gear pump from Micropump, model 187-000) at a rate of 50 mls/min (0.0061moles/min). 70% perchloric acid was fed by pump B (“GammaL”, diaphragm type, from Prominent, Inc.) at a rate of 4 gins/min (0.035 moles/min). The reactant streams were combined, reacted, crystallized, and quenched as in Example 1. The quench mixture was composed of 5 liters of methylene chloride, 1 liter of branched octane, and 3 liters of water. HPLC analysis of the product solution showed 0.4% DFCA, 98.2% Rofleponide-“R”, and 2.4% Rofleponide-“S”.

Branched octanes (2.5 liter) and water (2 liter) was then added to the product/quench mixture and stirred. The organic layer was then separated and treated with 16 gms of sodium bicarbonate in 2.1 liters of water, the pH of the final aqueous phase was 8. The organic phase was separated and distilled atmospherically to afford crude Rofleponide in 96% yield. HPLC analysis showed 0.24% DFCA, 98.67% Rofleponide-“R”, and 1.09% Rofleponide-“S”.

The crude material was recrystallized from acetone/n-heptane to afford Rofleponide final product in 67% yield. HPLC anaylsis showed 0.15% DFCA, 99.31% Rofleponide-“R”, and 0.54% Rofleponide-“S”.

1. A continuous process for the production of R-rofleponide that comprises the steps of:
a) mixing a continuous stream of dihydroflucinolone acetonide and butyraldehyde in a ratio of 1.5 to 5 moles of butyraldehyde per mole of dihydroflucinoline acetonide in a suitable solvent, with a continuous stream of aqueous perchloric acid having a concentration of 60 to 72 percent perchloric acid and having a flow rate such that there is, in the resulting mixture, a ratio of 2 to 6 moles of perchloric acid per mole of dihydroflucinolone acetonide;
b) allowing the mixture to flow through a reactor;
c) allowing the product to remain in a crystallizer for a suitable period of time;
d) removing the rofleponide perchloric acid complex from the crystallzier; and
e) recovering the rofleponide from the perchloric acid rofleponide complex.

2. A process according to claim 1 comprising the further step of recrystallizing R-rofleponide from a suitable solvent.

3. A process according to claim 1 in which the ratio of butyraldehyde to dihydroflucinoline acetonide is in the range of 2 to 4 moles of butyraldehyde per mole of dihydroflucinoline acetonide.

4. A process according to claim 1 in which the ratio of butyraldehyde to dihydroflucinoline acetonide is 3 moles of butyraldehyde per mole of dihydroflucinoline acetonide.

5. A process according to claim 1 in which the flow rate of the continuous stream of dihydroflucinolone acetonide and butyraldehyde and the flow rate of the continuous stream of aqueous perchloric acid are in a ratio such that the mole ratio of perchloric to dihydroflucinolone acetonide is in the range of 3 to 6 moles of perchloric acid per mole of dihydroflucinolone acetonide.

6. A process according to claim 1 in which the flow rate of the continuous stream of dihydroflucinolone acetonide and butyraldehyde and the flow rate of the continuous stream of aqueous perchloric acid are in a ratio such that the mole ratio of perchloric to dihydroflucinolone acetonide is in the range of 5 to 6 moles of perchloric acid per mole of dihydroflucinolone acetonide.

7. A process according to claim 6 in which the flow rate of the continuous stream of dihydroflucinolone acetonide and butyraldehyde and the flow rate of the continuous stream of aqueous perchloric acid are in a ratio such that the mole ratio of perchloric to dihydroflucinolone acetonide is in the range of 5 to 6 moles of perchloric acid per mole of dihydroflucinolone acetonide, and the ratio butyraldehyde to dihydroflucinoline acetonide is 3 moles of butyraldehyde per mole of dihydroflucinoline acetonide.

8. A process according to claim 7 in which the solvent is methylene chloride, the flow rate of the continuous stream of dihydroflucinolone acetonide and butyraldehyde and the flow rate of the continuous stream of aqueous perchloric acid are in a ratio such that the mole ratio of perchloric to dihydroflucinolone acetonide is in the range of 5 to 6 moles of perchloric acid per mole of dihydroflucinolone acetonide, and the ratio butyraldehyde to dihydroflucinoline acetonide is 3 moles of butyraldehyde per mole of dihydroflucinoline acetonide.

9. A process according to claim 1 in which the reactor is a pipe having an inside diameter in the range of 1/16 to 1 inch.

10. A process according to claim 1 in which the reactor is a pipe having an inside diameter is 1/16 inch.
